(19) United States
(12) Patent Application Publication
(10) Pub. No.: US 2024/0197624 A1
(43) Pub. Date: Jun. 20, 2024

(54) TIERED THERAPEUTIC RELEASE FROM A SINGLE RESERVOIR OF AN OPHTHALMIC DEVICE

(71) Applicant: TWENTY TWENTY THERAPEUTICS LLC, South San Francisco, CA (US)

(72) Inventors: Zidong Li, South San Francisco, CA (US); Christian Gutierrez, Pacifica, CA (US)

(21) Appl. No.: 18/539,046

(22) Filed: Dec. 13, 2023

Related U.S. Application Data
(60) Provisional application No. 63/432,411, filed on Dec. 14, 2022.

(51) Int. Cl. A61K 9/00; A61F 9/00

(52) U.S. Cl. CPC A61K 9/0051; A61F 9/0008; A61K 9/0009

(57) ABSTRACT

A tiered therapeutic reservoir of an ophthalmic device is described for controlled delivery of two or more therapeutic doses to a patient's eye. An ophthalmic device can include a reservoir having an interior and a metal electrode covering an opening of the reservoir. The interior of the reservoir can hold one or more pharmaceuticals or does of pharmaceuticals (referred to as drugs). The reservoir can hold a first drug and a second drug, with a sacrificial barrier layer positioned between the first drug and the second drug. The metal electrode can electrodissolve upon receiving an electrical signal to release the first drug from the reservoir. The sacrificial barrier layer can be sacrificed after the electrodissolution of the metal electrode to release the second drug from the reservoir. The first drug and the second drug can be released to an eye of a patient in contact with the ophthalmic device.

### RELATED APPLICATIONS

This application claims priority to U.S. Provisional Application Ser. No. 63/432,411, filed Dec. 14, 2022, entitled “TIERED THERAPEUTIC RELEASE FROM A SINGLE RESERVOIR OF AN OPHTHALMIC DEVICE”. The entirety of this application is hereby incorporated by reference for all purposes.

### TECHNICAL FIELD

The present disclosure relates generally to treatment of a disease/disorder of a patient's eye and, more specifically, to a tiered therapeutic reservoir of an ophthalmic device for controlled delivery of one or more therapeutics to the patient's eye to treat the disease/disorder.

### BACKGROUND

Diseases and disorders of a patient's eye can prove difficult to treat with one or more therapeutics delivered by traditional at home delivery methods. For example, the one or more therapeutics can be delivered as patient-controlled eye drops. However, this patent-controlled delivery of eye drops tends to leave the delivery of the one or more therapeutics subject to problems with precise positioning, dosing, and timing. Errors related to precise positioning and dosing can be eliminated with different delivery mechanisms, but the problems related to timing remain.

One therapeutic delivery mechanism employs an ophthalmic device, such as a contact lens placed directly over the eye, for delivery of the therapeutic to the eye. With an ophthalmic device, the therapeutic can be released into the eye in specific quantities and at specific target positions. However, precisely timing delivery of therapeutics remains an issue. Timing issues become apparent especially when one or more therapeutics need to be released on a specific schedule and/or in combination due to the limited size and space in which therapeutics can be stored.

### SUMMARY

In traditional ophthalmic devices, it has been impractical to have multiple drug reservoirs timed for specific teared release of one or more therapeutics. The present disclosure provides a multi-tiered reservoir within an ophthalmic device that can control release of one or more therapeutics from the multi-tiered reservoir for the delivery of one or more therapeutics to treat a disorder of the eye.

In an aspect, the present disclosure includes an ophthalmic device that can deliver at least one therapeutic, such as at least one drug, to an eye of a subject from a multi-tiered reservoir. The ophthalmic device comprises a reservoir having an interior and a metal electrode configured to cover an opening of the reservoir. The interior of the reservoir comprises a first drug, a sacrificial barrier layer, and a second drug, where the sacrificial barrier layer is located between the first drug and the second drug. The metal electrode is configured to receive an electrical signal that electrodissolves the metal electrode. Upon electrodissolution of the metal electrode the first drug is released. The second drug is released upon sacrifice of the sacrificial barrier layer, a time after the electrodissolution of the metal electrode.

In another aspect, the present disclosure includes a method for releasing at least one therapeutic, such as at least one drug, to an eye of a patient from a multi-tiered reservoir of an ophthalmic device. The method includes electrodissolving a metal electrode covering an opening of a reservoir, the multi-tiered reservoir, of the ophthalmic device. A drug is then released from the opening of the reservoir. After the drug is released a sacrificial barrier layer that holds a second drug in the reservoir is sacrificed. Then after the sacrificial barrier layer is sacrificed the second drug is released from the opening of the reservoir.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 shows a diagram of an ophthalmic device that can deliver a therapeutic to a subject's eye;

FIG. 2 shows a magnified portion of the multi-tiered reservoir of FIG. 1;

FIG. 3 shows time-lapse diagrams of therapeutic release from the multi-tiered reservoir of FIG. 1;

FIG. 4 shows a generic diagram of a multi-tiered reservoir of an ophthalmic device;

FIG. 5 shows time-lapse diagrams of therapeutic release from a multi-tiered reservoir having two sacrificial barrier layers;

FIG. 6 shows a graphical representation of drug releases for a multi-tiered reservoir having various thicknesses of two sacrificial barrier layers;

FIG. 7 shows an exemplary ophthalmic device in relation to the eye;

FIG. 8 shows an exemplary ophthalmic device comprising multiple multi-tiered reservoirs; and

FIGS. 9 and 10 are process flow diagrams illustrating methods for controlling the release of multiple therapeutics from an ophthalmic device.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “ophthalmic device” refers to a medical instrument used on or within a portion of a patient's eye for optometry or ophthalmology purposes (e.g., for diagnosis, surgery, vision correction, or the like). An ophthalmic device can be “smart” when it includes one or more components that facilitate one or more active processes for purposes other than traditional lens-based vision correction (e.g., therapeutic release). Unless otherwise stated, as used herein, the term “ophthalmic device” should be understood to mean “smart ophthalmic device”.

As used herein, the term “reservoir” refers to a storehouse for a therapeutic. The reservoir can be encapsulated within a body of an ophthalmic device, which may comprise a hydrogel based material. A portion of the reservoir can be open for release of the therapeutic (allowing for diffusion of the therapeutic out of the reservoir and into the surrounding hydrogel matrix). The opening may be covered to prevent release of the therapeutic. In some instances, the covering can be used to control the release of the therapeutic from the reservoir.

As used herein, the term “therapeutic” refers to one or more substance (e.g., liquid, solid, or gas) related to the treatment, symptom relief, or palliative care of a disease, injury, or other malady. Examples of the therapeutic can include one or more drugs, other types of pharmaceuticals, or the like.

As used herein, the term “electrode” refers to an at least partially conductive solid (e.g., including one or more metals, one or more polymers, or the like) that receives/transmits an electrical signal. A non-limiting example of an electrode is a thin-film gold electrode.

As used herein, the term “electrical signal” refers to a signal waveform generated by an electronic means, such as a generator. An electrical signal may be a voltage signal, a current signal, or the like.

As used herein, the term “electrodissolution” refers to a process for dissolving a solute using an electrical catalyst. In one non-limiting example, application of an electrical signal to a solid metal can cause the solid metal to dissolve into separate molecules, such as when an electrical signal with proper parameters is delivered to an electrode covering an opening of a reservoir such that the electrode electrodissolves.

As used herein, the term “hydrogel-based material” refers to a soft contact lens material, such as a hydrogel or a silicone-hydrogel material, including, but not limited to, all hydrogel and silicone-hydrogel materials. Other materials that may be used in a soft contact lens are also included as or within a hydrogel-based material.

As used herein, the term “hydrogel” refers to a crosslinked hydrophilic polymer that does not dissolve in water. A hydrogel is generally highly absorbent yet maintains a well-defined structure.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

### II. Overview

Traditional treatments of a disease/disorder of a patient's eye include or more therapeutics delivered manually via eye drops, but manual delivery suffers from problems related to precise positioning, dosing, and timing. Ophthalmic devices positioned in or on the eye have emerged as automatic therapeutic delivery mechanisms that can provide precise positioning and dosing, but such ophthalmic devices still suffer from problems related to precisely timing delivery of the doses, especially when a dose needs to be administered at a very specific time relative to another dose. Scheduling electronically mediated release of a therapeutic in an appropriate dose at a specific time, and then doing the same with another therapeutic (or another dose of the same therapeutic) in another reservoir poses difficulties, including the need for precise timing control and additional bulky circuitry inside the ophthalmic device. The disclosure gets around these problems with delivery timing by implementing a single reservoir with multiple tiers that achieves tiered therapeutic release for controlled delivery to the patient's eye to treat the disease/disorder.

A multi-tiered reservoir within an ophthalmic device can be utilized to release one or more therapeutics of the same or different compositions on a precise schedule and in specific dosages. The one or more therapeutics can be held in an interior of a reservoir of the ophthalmic device and separated by a sacrificial layer of a thickness. An opening of the reservoir is covered by a single metal electrode (e.g., made of gold or copper). For each additional therapeutic in the reservoir, an additional sacrificial barrier layer can be used to separate two therapeutics. The first therapeutic can be released from the ophthalmic device by dissolving the metal electrode using an electrical signal as a catalyst to drive an electrodissolution of the metal electrode. The second therapeutic, and any additional therapeutics, can be released from the ophthalmic device by sacrificing the sacrificial barrier layer(s) (e.g., by passive dissolution due to solubility, active electrophoresis, etc.) with a dissolution time based on at least the material composition and the thickness of the sacrificial barrier layer. A multi-tiered reservoir can thus significantly save space and simplify the circuitry and control complexities of multiple therapeutic release for combinatorial or extended therapeutic release protocols.

### III. Systems

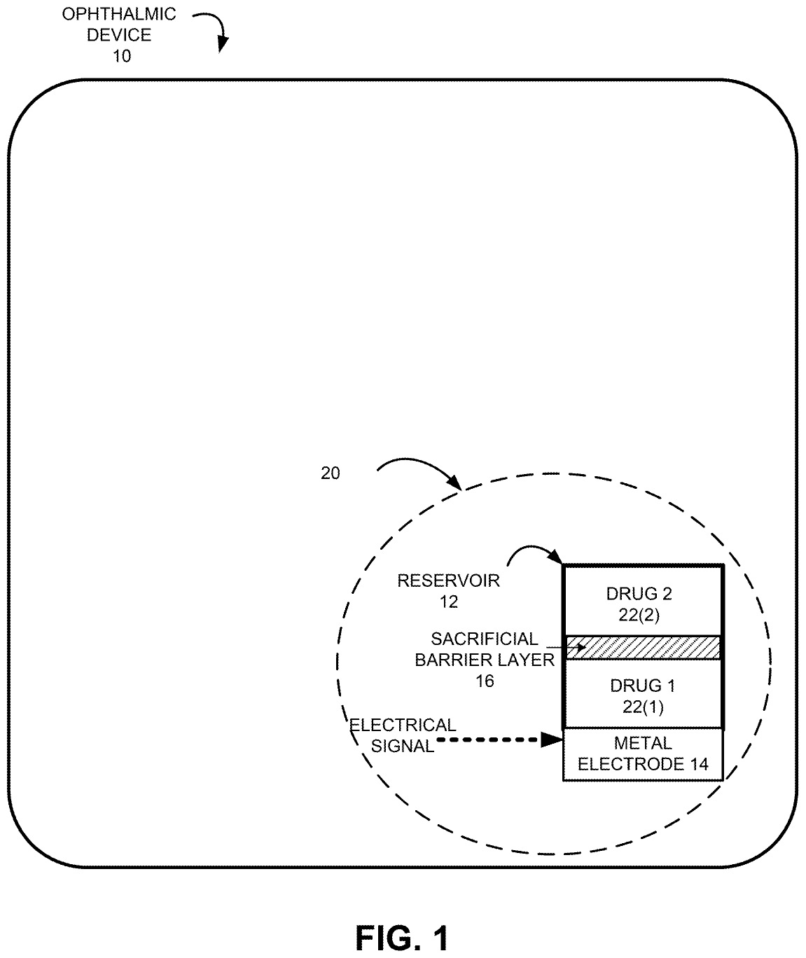

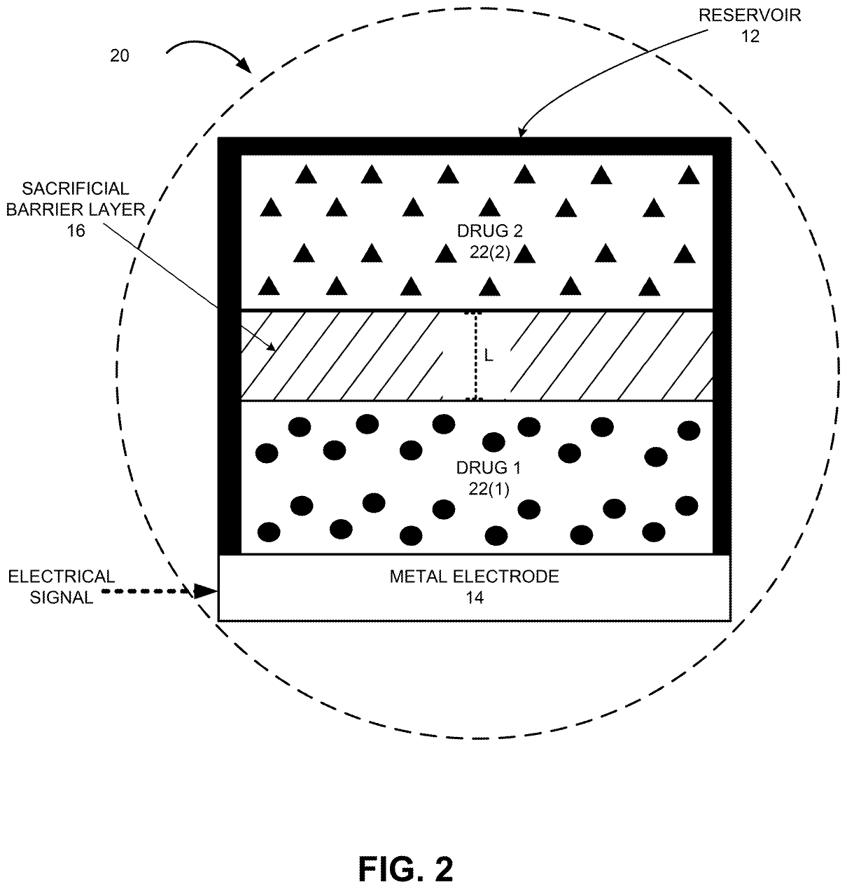

As shown in FIG. 1, one aspect of the present disclosure includes an ophthalmic device 10 that includes a multi-tiered reservoir system 20 (described in more detail with respect to FIG. 2) for therapeutic release. The ophthalmic device 10 can be, for example, a contact lens that can be positioned on the surface of an eye, an eye implant, or any other ophthalmic device that can be in contact with at least a portion of the eye of a patient. The ophthalmic device 10 can be used, for example, for combinatorial therapeutic dosage protocols, where two or more different therapeutics are delivered into the eye for improved efficacy of treatment. In another example, the ophthalmic device 10 can be used for extended release of a single therapeutic, within a preferred dosage. The multi-tiered reservoir system 20 can be encapsulated within a body of the ophthalmic device 10. The reservoir 12 can be shaped to hold one or more therapeutics (e.g., Drug 1 and Drug 2 22(1) and (2)) and one or more sacrificial barrier layers 16 and sized to fit within the volume of the body of the ophthalmic device 10 (for example, the reservoir can have a diameter on the order of tens or hundreds of microns, such as 5 μm, 50 μm, or 500 μm). The interior of the reservoir 12 can be configured to hold the one or more therapeutic in at least one of liquid, solid, and gas states. The one or more therapeutics can be, for example, used for the treatment and/or symptom relief of diseases such as glaucoma and dry eye. The reservoir 16 can be made of photo-patternable polymers such as an epoxy-based negative photoresist material (SU-8), a positive photoresist material (AZ 1500), a cyclic olefin copolymer (COC), a cyclic olefin polymer (COP), or other thermoplastic polymers such as liquid crystal polymer (LCP), Parylene, Polyimide, polypropylene, polycarbonate, Ultem or Nylon.

The multi-tiered reservoir system 20 can achieve precise positioning, dosing, and timing of the release of one or more therapeutics (e.g., drugs, pharmaceuticals, etc.) to treat and/or relieve symptoms of diseases, disorders, or injuries of the eye. The multi-tiered reservoir system 20 includes a reservoir 12 having an opening covered by a metal electrode 14 that is subject to electrodissolution by receiving an electrical signal. The reservoir 12 can hold a plurality of therapeutics (represented as Drug 1 22(1) and Drug 2 22(2), anything referred to as a “drug” herein refers to a “therapeutic” more broadly) separated by a sacrificial barrier layer 16. Although shown positioned in the bottom right of the ophthalmic device 10 in FIG. 1, it should be understood that the multi-tiered reservoir system 20 may be placed anywhere within the ophthalmic device where reservoirs are traditionally placed to deliver one or more therapeutics to a patient's eye and may be of varying size and shape. Additionally, drugs 22(1) and 22(2) may be different therapeutics or drugs 22(1) and 22(2) may be the same therapeutic. The therapeutic(s) each be stored in, for example, a solid state, a liquid state, or a gaseous state as known in the art.

The ophthalmic device 10 can include one reservoir 12 (shown in FIG. 1), however the ophthalmic device may include any number of reservoirs, as discussed in more detail below. The reservoir 12 can be positioned within the ophthalmic device 10 in any manner conducive to releasing therapeutics to an eye the ophthalmic device is in/on. It should be noted that FIG. 1 (and subsequent figures) show the interior of the reservoir for explanatory purposes, but the reservoir is meant to only have the one opening to the interior covered by the metal electrode 14. For example, the reservoir 12 can be positioned with the metal electrode 14, positioned facing the eye and outside of the eye's line of vision.

As shown in FIG. 2, the multi-tiered reservoir system 20 can include a reservoir 12 having an interior and an opening to the interior that is covered by a metal electrode 14. The metal electrode 14 can cover the opening to the interior of the reservoir 12. The metal electrode 14 can include one or more electrochemically active metals. One example of such a metal is gold. The gold can be thin enough to facilitate the electrodissolution (and in some instances, the length of time to achieve electrodissolution can be based on the thickness of the gold), like the non-limiting example of a gold film electrode. Other non-limiting examples of electrochemically active metals include silver, platinum, and copper. The metal electrode 14 can electrodissolve when an electrical signal is applied to the metal electrode from a signal generator (not shown in FIG. 2). The electrodissolution of the metal electrode 14 may be facilitated by the presence of chloride ions from tears from the eyes and/or packaged within the ophthalmic device 10. The interior of the reservoir 12 can include at least a first drug (Drug 1) 22(1), a sacrificial barrier layer 16, and a second drug (Drug 2) 22(2).

The sacrificial barrier layer 16 can be positioned within the interior of the reservoir 12 to create separate compartments (e.g., tiers) that can each hold a quantity of a therapeutic (e.g., Drug 1 22(1) and Drug 2 22(2)). As shown, Drug 1 22(1) and Drug 2 22(2) can be within the reservoir 12 and in compartments separated by a sacrificial barrier layer 16. A compartment for Drug 1 22(1) can be formed between the metal electrode 14 and the sacrificial barrier layer 16 form, while Drug 2 22(2) can be within another compartment separated by the sacrificial barrier layer 16 and the end of the reservoir 12. Drug 1 22(1), can be released upon at least partial electrodissolution of the metal electrode 14 (designed based on one or more properties of the metal electrode 13. Drug 2 22(2) can be released upon the at least partial degradation (e.g., passive and/or active) of the sacrificial barrier layer 16 (based on one or more properties of the sacrificial barrier layer 16, such as the thickness L, the contents of the sacrificial battier layer, etc.).

For example, as shown in FIG. 2, the sacrificial barrier layer 16 can be positioned between the first drug (Drug 1) 22(1) and the second drug (Drug 2) with the first drug (Drug 1) positioned nearer the opening of the reservoir and the metal electrode 14 so that the first drug (Drug 1) is released and/or delivered before the second drug (Drug 2). The metal electrode 14 can cover the opening of the reservoir 12 until it is removed, such as by electrodissolution (e.g., caused by application of an electrical signal to the electrode 14). An electrical signal (e.g., from a generator (not shown) and controlled by a controller (not shown), which may be at least partially within the ophthalmic device) can be applied to the metal electrode 14, on demand or based on one or more inputs (e.g., physiological parameters, time, etc.). The metal electrode 14 can receive the electrical signal and can begin to electrodissolve. The electrical signal may be delivered to the metal electrode 14 until the metal electrode has partially or fully electrodissolved. The first drug (Drug 1) 22(1) can be released from the reservoir 12 upon the electrodissolution (partial or complete) of the metal electrode 14. The first drug (Drug 1) 22(1) can passively and/or actively (e.g., facilitated by iontophoresis—the passing of an electrical signal between at least two electrodes to increase the speed of movement of the molecules of the therapeutic) diffuse out of the reservoir 12 and out of the ophthalmic device 10 upon release from the reservoir. Then, after a period of time (which can be selected/chosen based on an efficacy profile of the therapeutic, a prescribed timing for optimal therapeutic interactions, a prescribed timing for no therapeutic interactions, etc. depending on the circumstance), the sacrificial barrier layer 16 can be sacrificed and the second drug (Drug 2) 22(2) can be released upon the sacrifice (partial or complete) of the sacrificial barrier layer. The time can be based on the materials used in the sacrificial barrier layer 16, the thickness (L), and/or any other property chosen for the reservoir. The sacrifice can be, for example, water mediated or electrically mediated depending on the composition of the sacrificial barrier layer 16. The second drug (Drug 2) 22(2) can passively and/or actively (e.g., facilitated by iontophoresis) diffuse out of the reservoir 12 and out of the ophthalmic device 10 upon release from the reservoir.

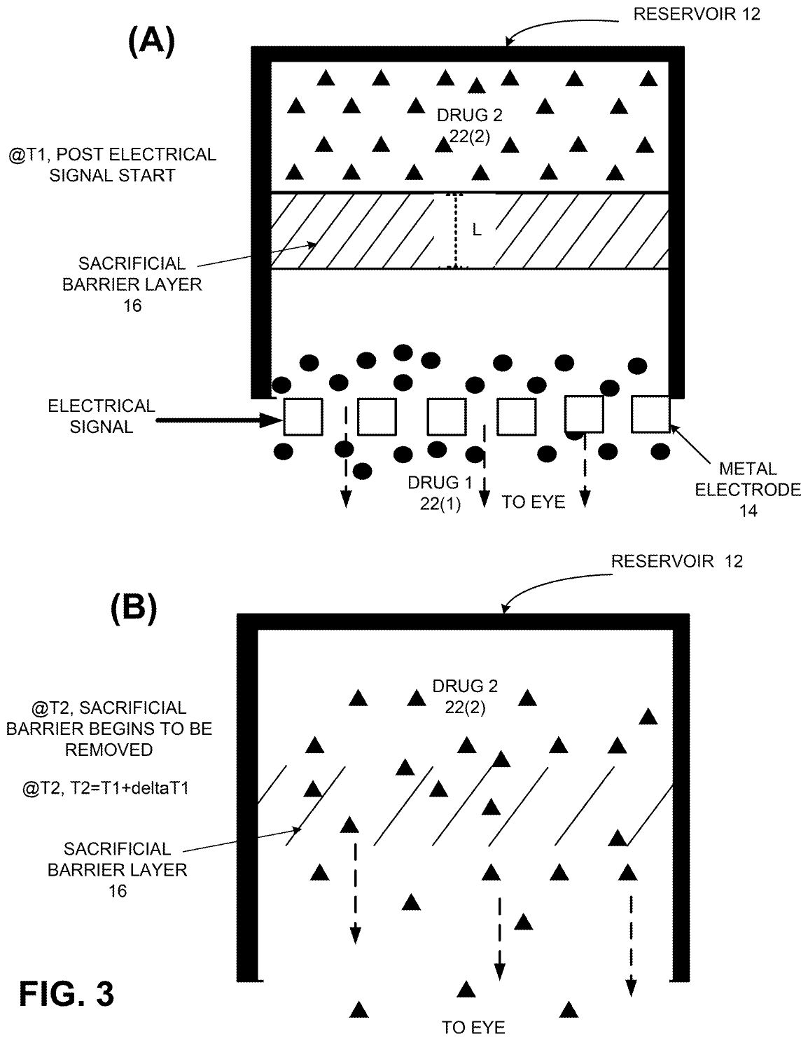

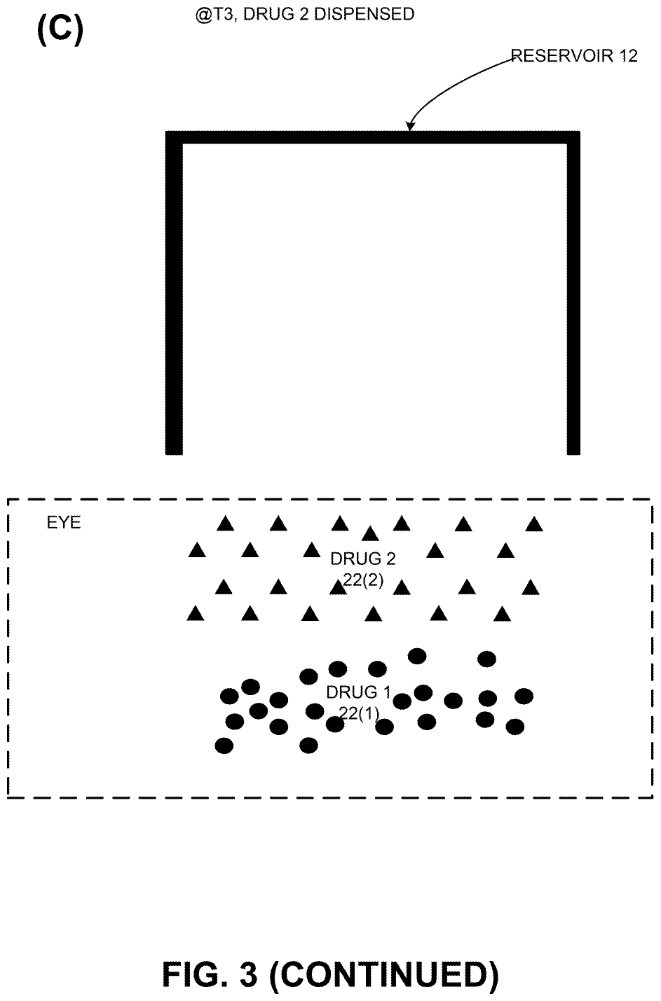

FIG. 3 shows an example of the use of the multi-tiered reservoir system 20 to deliver two therapeutics into an eye for treatment and/or relief of a disease, disorder, or injury. As shown in FIG. 3, element A, at time T₁ after the electrical signal starts and once the metal electrode 14 has begun to electrodissolve (due to the application of the electrical signal) the therapeutic, shown as Drug 1 22(1), can be released from the compartment created in the interior of the reservoir 12 between the sacrificial barrier layer 16 and the metal electrode. After Drug 1 22(1) is released, Drug 1 can passively (e.g., via diffusion) or actively (e.g., via iontophoresis or other means) move out of the reservoir 12, and the ophthalmic device 10 (not shown in FIG. 3) towards the eye. The time Drug 1 22(1) takes to move to the eye can depend on factors that include, but are not limited to, the speed and quantity of the electrodissolution of the metal electrode 12, the material composition of the body of the ophthalmic device, the position of the reservoir 12 within the ophthalmic device relative to the eye, etc.

As shown in FIG. 3, element B, at a time T₂ following the release of the therapeutic (e.g., Drug 1 22(1)) stored between the metal electrode 14 and the sacrificial barrier layer 16 the sacrificial barrier layer can begin to be sacrificed. When the sacrificial layer 16 has begun to be sacrificed the therapeutic (Drug 2 22(2)) stored between the sacrificial barrier layer and the bottom of the interior of the reservoir 12 can be released towards the eye. The time T₂ can be a predetermined time ΔT after T₁. The predetermined time ΔT can be based, for example, on the composition of the sacrificial barrier layer 16, the thickness (L) of the sacrificial barrier layer 16, and/or the method by which the sacrificial barrier layer 16 is sacrificed (e.g., passive dissolution due to solubility, active electrophoresis, etc.). For example, the sacrificial barrier layer can include or be composed of a water soluble material to facilitate the sacrifice as the electrodissolution of the metal electrode 14 and the release of the therapeutic Drug 1 22(1) allow water molecules, such as saline from tears of the eye, to diffuse into the ophthalmic device. The water soluble material can then begin to break down when in the presence of the water molecules. The water soluble material can include, for example, a water soluble polymer and/or a water soluble salt. Water soluble materials include, but are not limited to, Polyvinyl alcohol (PVA), Polyvinylpyrrolidone (PVP), Polyethylene glycol (PEG), or Polyacrylic acid (PAA).

In another example, the sacrificial barrier layer 16 can be electronically activated, via another electrical signal, to facilitate the sacrifice. The sacrificial barrier layer 16 can include a metal configured to undergo electrodissolution when the other electrical signal is applied. The other electrical signal may be the same as the electrical signal that electrodissolved the metal electrode 14 or different. If the other electrical signal is different, then it may have the same parameters as the electrical signal or different parameters. The sacrificial barrier layer 16 can also, or alternatively includes one or more electroresponsive polymers and/or one or more electrothermal films that can be activated by the other electrical signal. Once released by the sacrifice of the sacrificial barrier layer 16 the therapeutic (Drug 2 22(2)) can passively (e.g., via diffusion) or actively (via iontophoresis or other means) move out of the reservoir 12, and the ophthalmic device 10 (not shown in FIG. 3) towards the eye. The time Drug 2 22(2) takes to move to the eye can depend on factors that include, but are not limited to, the quantity and speed of the sacrifice of the sacrificial barrier layer 16 (as a function of how much and how quickly the sacrificial barrier layer is dissolved), the material composition of the body of the ophthalmic device, the position of the reservoir 12 within the ophthalmic device relative to the eye, etc. For example, release profile of Drug 2 22(2) can be based on the thickness of the sacrificial barrier layer 16.

As shown in FIG. 3, element C, at time T₃ both Drug 1 22(1) and Drug 2 22(2) can be dispensed to the eye from the reservoir 12 (ophthalmic device not shown). Drug 1 22(1) and Drug 2 22(2) can be the same or different therapeutics. For example, Drug 1 22(1) and Drug 2 22(2) can be different drugs that can interact when combined after both drugs have been released, where ΔT is short enough that both drugs can be in the eye and/or the ophthalmic device at overlapping times. In another example Drug 1 22(1) and Drug 2 22(2) can be different drugs that are not considered combinable so the time ΔT can be long enough for D1 to have dispersed within the eye. In another example, Drug 1 22(1) and Drug 2 22(2) can be the same therapeutic that is released into the eye over an extended period of time to maximize the efficacy curve of the therapeutic without delivering too large a bolus at once.

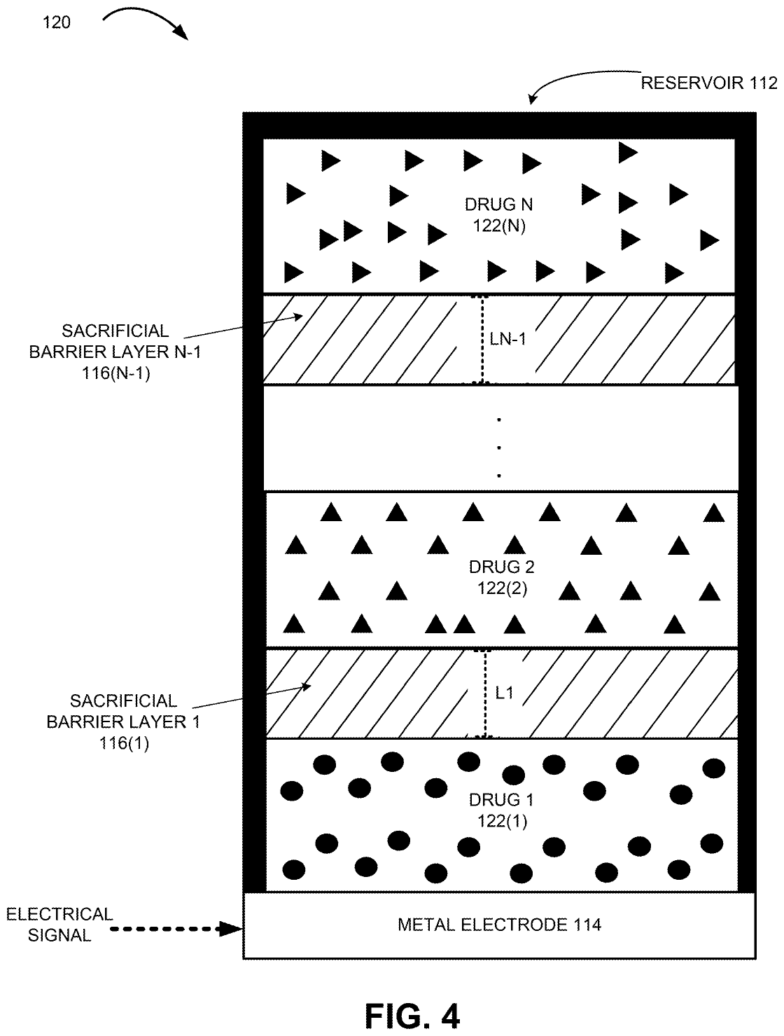

FIG. 4 shows a generic example of a multi-tiered reservoir system 120 having more tiers than the two tiers shown in FIGS. 1-3. The reservoir 112 can have an interior that can include a number of sacrificial barrier layers 116(1)-116(N−1), where N is an integer greater than or equal to three, and a number of tiers for holding the therapeutics Drug 1 122(1)-Drug N 122(N), where N is an integer greater than or equal to three. An opening to the interior of the reservoir 112 can be covered by the metal electrode 114, which can be electrodissolved with the application of an electrical signal. For example, if N=3, then the interior of the reservoir 112 can include a second sacrificial barrier layer 116(2) and a third therapeutic Drug 3 122(3). Then, the Drug 3 122(3) can be released upon the sacrifice of the second sacrificial barrier layer 116(2) a second predetermined time after the first sacrificial barrier layer 116(1) was sacrificed. The number of sacrificial barrier layers 116 (N−1) and the number of tiers that can store therapeutics 122(N) can be based on the size of the ophthalmic device and the reservoir, the quantities and/or state (e.g., solid, liquid, or gas) of the therapeutics, the thicknesses of the sacrificial layers (which may be the same or different) and/or on the desired therapeutic release profiles for treatment purposes.

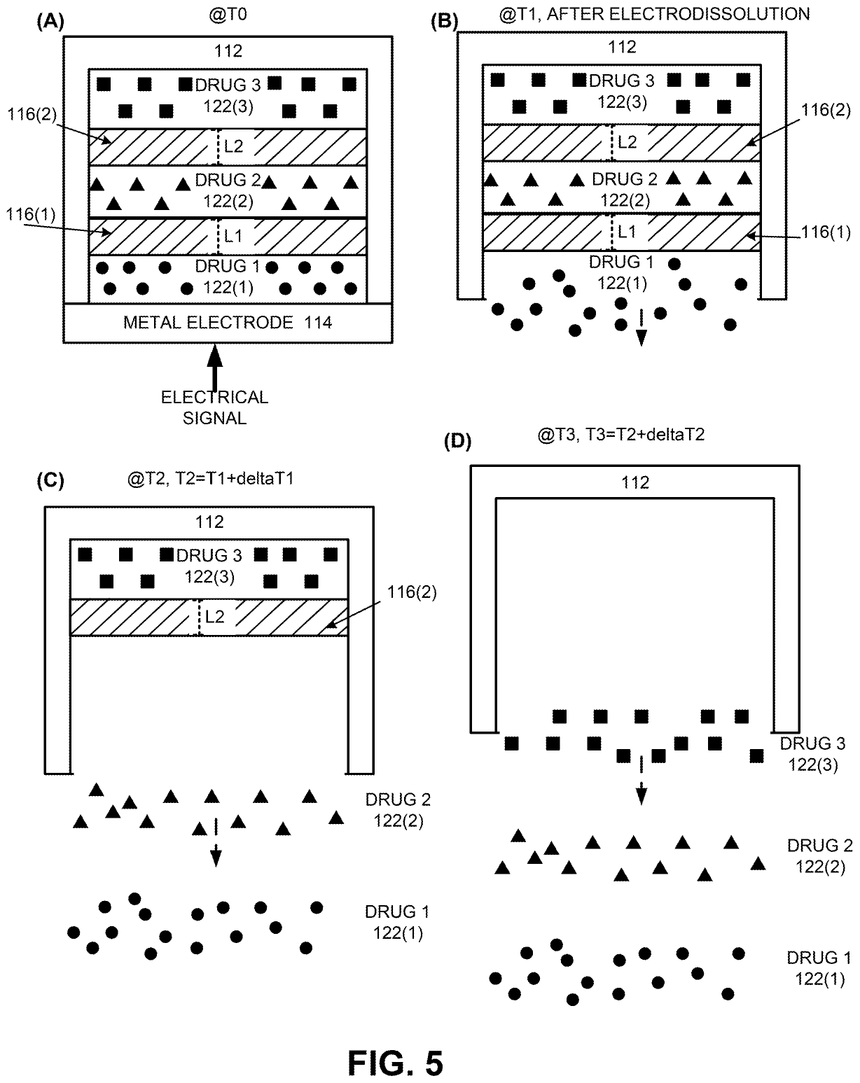

FIG. 5, elements A-D, illustrate the release of therapeutics from a reservoir 112 of a three-tiered reservoir system of an ophthalmic device. The interior of reservoir 112 can have an opening covered by the metal electrode 114 and can include Drug 1 122(1) stored between the metal electrode 114 and the first sacrificial barrier layer 116(1), the first sacrificial barrier layer, Drug 2 122(2) stored between the first sacrificial barrier layer and the second sacrificial barrier layer 116(2), the second sacrificial barrier layer, and Drug 3 122(3) stored between the second sacrificial barrier layer and a bottom of the interior of the reservoir. FIG. 5, element A shows time T₀, the start of the application of the electrical signal to the metal electrode 114. The metal electrode 114 can electrodissolve in response to the application of the electrical signal. As shown in FIG. 5, element B, at T₁ after the electrodissolution of the metal electrode 114 the Drug 1 122(1) can be released from the reservoir and can move through the ophthalmic device (not shown in FIG. 5) to the eye (not shown in FIG. 5). Drug 1 122(1) can move passively (e.g., via diffusion) and/or actively (e.g., via iontophoresis). Then the first sacrificial layer 116(1) can be sacrificed.

As shown in FIG. 5, element C, at time T₂ the first sacrificial layer 116(1) can be sacrificed and Drug 2 122(2) can be released from the reservoir 112. Time T₂ can be a predetermined time ΔT after T₁. The predetermined time ΔT can be based on, for example, the thickness of the first sacrificial barrier layer 116(1), the composition of the first sacrificial barrier layer, and/or the method of sacrifice of the first sacrificial barrier layer. After release, Drug 2 122(2) can move passively (e.g., via diffusion) and/or actively (e.g., via iontophoresis) towards the eye. After the release of Drug 2 122(2), the second sacrificial barrier layer 116(2) can be sacrificed. As shown in FIG. 5, element D, at time T₃ the second sacrificial barrier can have been sacrificed and Drug 3 122(3) can be released from the reservoir 112. Time T₃ can be a predetermined time ΔT₂ after T₂. The predetermined time ΔT₂ can be based on, for example, the thickness of the second sacrificial barrier layer 116(2), the composition of the second sacrificial barrier layer, and/or the method of sacrifice of the second sacrificial barrier layer. After release, Drug 3 122(3) can move passively (e.g., via diffusion) and/or actively (e.g., via iontophoresis) towards the eye. While FIG. 5 shows sequential sacrifice of the sacrificial barrier layers after the metal electrode has been dissolved in another example one or more of the sacrificial barrier layer(s) can be sacrificed before the electrodissolution of the metal electrode to mix one or more of the drugs together before they are released towards the eye.

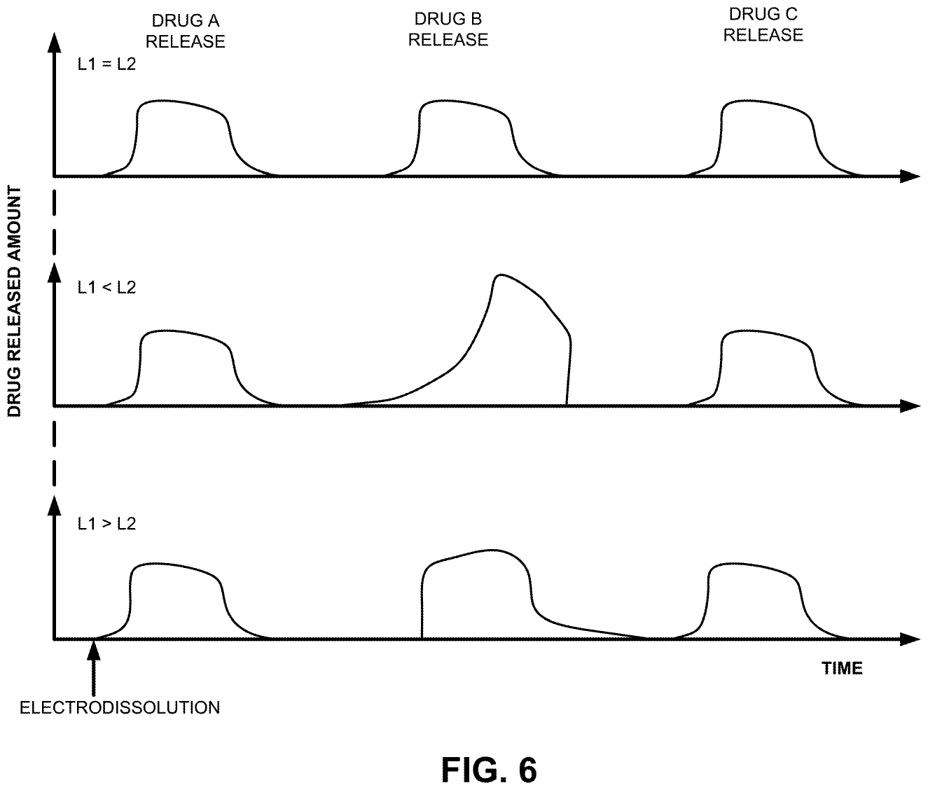

FIG. 6 illustrates graphical representation of therapeutic release profiles of three drugs (Drug A, Drug B, and Drug C, each of which could be Drug 1, Drug 2, and/or Drug 3) with varying thicknesses (L) of a first water soluble sacrificial barrier layer compared to a second water soluble sacrificial barrier layer with the same thickness throughout. Where Drug A is held between a metal electrode and a first sacrificial barrier layer having a thickness L1, Drug B is held between the first sacrificial barrier layer having thickness L1 and a second sacrificial barrier layer having thickness L2, and Drug C is held between the second sacrificial barrier layer having thickness L2 and the bottom of the interior of a reservoir. In the top graphical layer of FIG. 6 the thickness L1 of the first sacrificial barrier layer and the thickness L2 of the second sacrificial barrier layer are equal and the drug release profiles (amounts over time) are the same and/or similar for each of Drug A, B, and C after release. In the middle graphical layer of FIG. 6 the thickness L1 of the first sacrificial barrier layer is less than the thickness L2 of the second sacrificial barrier layer and the drug release profile (amount over time) of Drug B and Drug C differ, with more of Drug B being released sooner compared to when the thicknesses were the same. In the bottom graphical layer of FIG. 6 the thickness L1 of the first sacrificial barrier layer is greater than the thickness L2 of the second sacrificial barrier layer and the drug release profile (amount over time) of Drug B and Drug C differ, with Drug B taking longer to begin release compared to when the thicknesses were the same or L1 was less than L2. The release profiles of Drugs A and C are unchanged as there is no change to the electrodissolution of the metal electrode to release Drug A and the thickness of L2 remains constant (the thickness of L1 is varied).

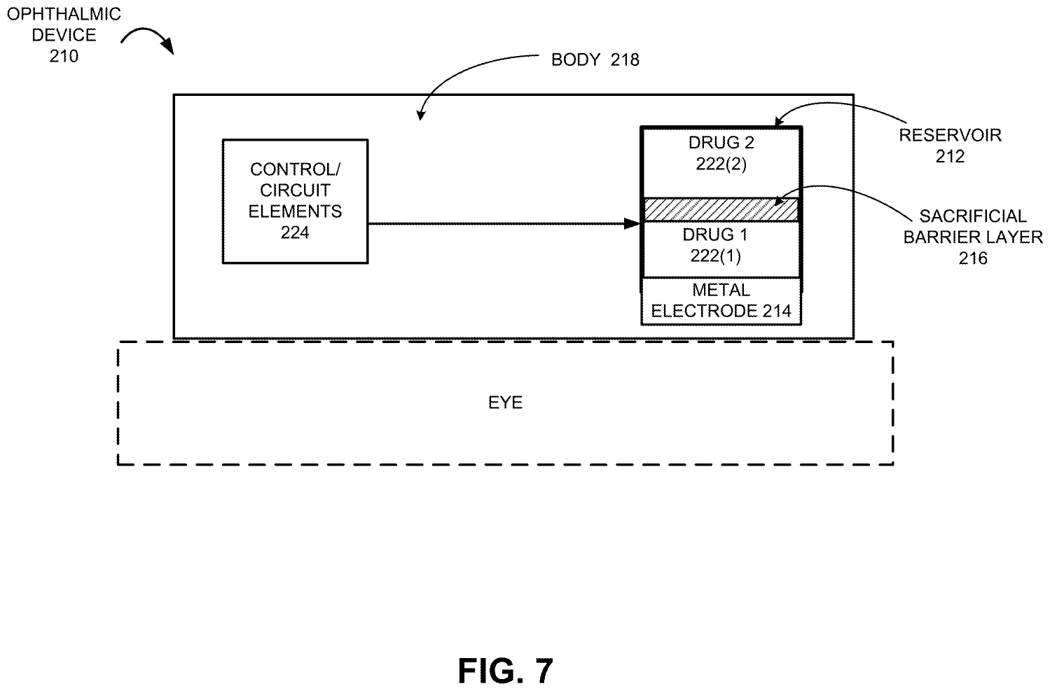

FIG. 7, shows an example ophthalmic device 210, not shown to scale or shape. Ophthalmic device 210 can be, for example, a contact lens placed on a surface of an eye. The ophthalmic device 210 can have a body 218 that fully encapsulates the reservoir 212 and the metal electrode 214 as well as additional control and circuit elements 224. The body 218 of the ophthalmic device can be made of a hydrogel matrix formed of a hydrogel-based material and water. The hydrogel-based material can be any cross-linked hydrophilic polymer that does not dissolve in water. Accordingly, the hydrogel-based material can be stiff when dry, but soft and pliable when hydrated. The hydrogel-based material is highly absorbent and has a naturally-high water content (e.g., 20%-60%), yet maintains a well-defined structure. Non-limiting examples of hydrogel-based material monomers are hydroxyethylmethacrylate (HEMA) or derivatives, methacrylic acid (MA) or derivatives, methyl methacrylate (MMA) or derivatives, n-vinyl perrolidone (NVP) or derivatives, poly vinyl alcohol (PVA) or derivatives, polyvinyl pyrrolidone (PVP) or derivatives, and the like. In some instances, the hydrogel-based material can include silicone (as a “silicone-hydrogel”), increasing the oxygen transmissibility and permeability of the hydrogel (among other bulk and surface properties that the presence of silicone improves).

The reservoir 212 is shown as a two-tiered reservoir having an opening to an interior covered by metal electrode 214, but can include any number of tiers, and having an interior that can include a first therapeutic (Drug 1 222(1)), a sacrificial barrier layer 216, and a second therapeutic (Drug 2 222(2)). The reservoir 212 can be shaped to hold the therapeutics 222(1) and (2) and the sacrificial barrier layer 216 and sized to fit within the volume of the body 218 (for example, the reservoir can have a diameter on the order of tens or hundreds of microns, such as 5 μm, 50 μm, or 500 μm). The therapeutics 222(1) and 222(2) can be the same or different and can be a liquid, solid, or gas. The therapeutics 222(1) and 222(2), for example, can be used for the treatment and/or symptom relief of diseases such as glaucoma and dry eye. The reservoir 212 can be made of photo-patternable polymers such as an epoxy-based negative photoresist material (SU-8), a positive photoresist material (AZ 1500), a cyclic olefin copolymer (COC), a cyclic olefin polymer (COP), or other thermoplastic polymers such as liquid crystal polymer (LCP), Parylene, Polyimide, polypropylene, polycarbonate, Ultem or Nylon. The metal electrode 214 can include one or more electrochemically active metal. One example of such a metal is gold. The gold can be thin enough to facilitate the electrodissolution, like the non-limiting example of a gold film electrode. The reservoir 212 can be oriented such that the metal electrode 214 covered opening is facing towards the eye.

One or more of the control/circuit elements 224 can be in electrical communication with at least one portion of the reservoir 212 or the metal electrode 214. Examples of control/circuit elements 224 can include, but are not limited to, a signal generator, a power source, circuitry, and a microcontroller. For example, control circuit elements 224 can include a signal generator, and a microcontroller that can configure and transmit (via circuitry) an electrical signal (which can be a current signal and/or a voltage signal) to at least the metal electrode 214. In some instances, another electrical signal (which may be the same or different) can be applied to the sacrificial barrier layer 216 as described above. The control/circuit elements 224 can also facilitate the active movement of therapeutics 222(1) and/or 222(2) after release via iontophoresis when the control/circuit elements include one or more iontophoresis electrodes too.

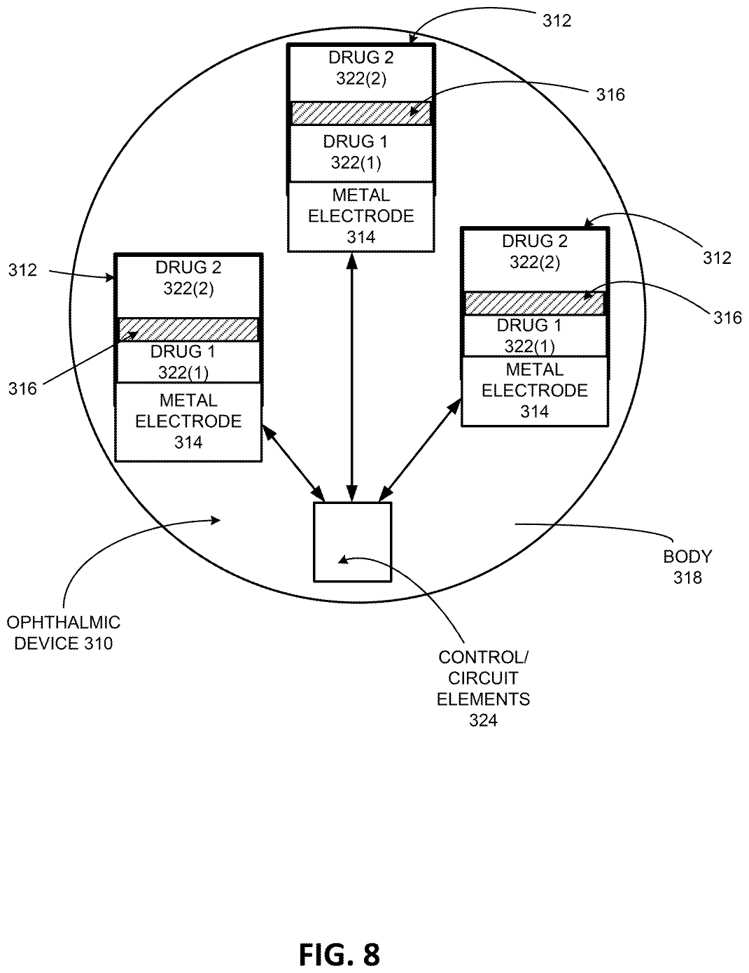

FIG. 8 shows another example of an ophthalmic device 310. In FIG. 8, the ophthalmic device 310 is shown representing a contact lens, but it should be understood that the ophthalmic device 310 may be any other type of ophthalmic device. Ophthalmic device 310 is shown as circular but can be any shape. The ophthalmic device 310 has a body 318 that can fully encapsulate a plurality of multi-tiered reservoir systems (shown as reservoirs 312 and metal electrodes 314). Three multi-tiered reservoir systems are shown, but any number one or greater can be encapsulated in the body of the ophthalmic device 310 as necessary for a specific treatment. Control/circuit elements 324, as described in FIG. 7 as 224) can be in electrical communication with each of the plurality of multi-tiered reservoirs (wires can run in any way through the ophthalmic device). Communication may be bi-directional. Each of the multi-tiered reservoir systems has a reservoir 312 having an interior with an opening covered by metal electrode 314. The interior of each of the reservoirs 312 can include at least one sacrificial layer 316 that creates a compartment to hold a Drug 1 322(1) (between the metal electrode 314 and the sacrificial barrier layer) and another compartment to hold a Drug 2 322(2) (between the bottom of the interior of the reservoir and the sacrificial barrier layer). The drugs of each multi-tiered reservoir system can be released as described above with respect to FIGS. 1-7 at specified times. For example, different reservoirs can include different or the same drugs that can have timed and/or controlled releases throughout a given time cycle (e.g., an hour, a day, a week, a month, etc.). Each of the reservoirs 312 can be positioned outside of the viewing area of the eye so as to not disrupt a patient's vision.

### IV. Methods

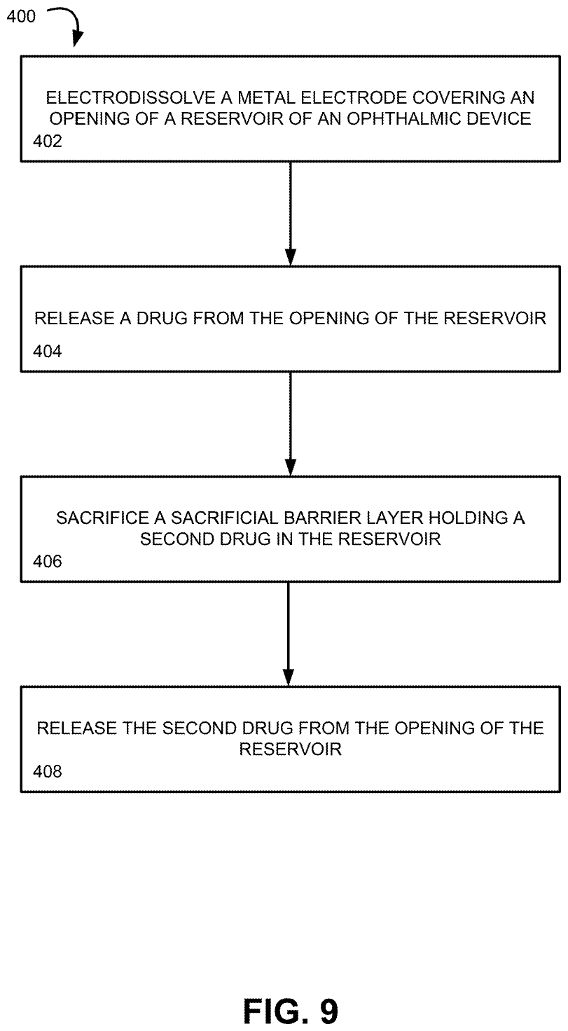

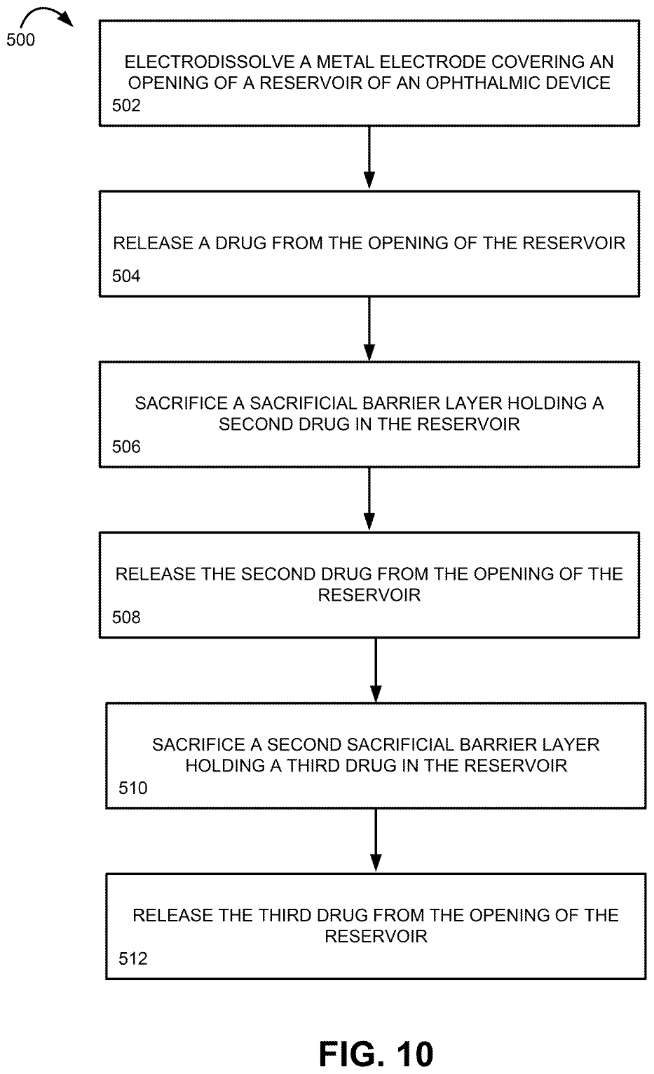

Another aspect of the present disclosure can include methods 400 and 500 (FIGS. 9 and 10) for controlling the release of at least one therapeutic from an ophthalmic device to treat a disease, disorder, or injury of an eye wearing/containing the ophthalmic device. The methods 400 and 500 can be executed using, at least, the ophthalmic device 10 and the multi-tiered reservoir system 20 (or iterations thereof) described above with respect to FIGS. 1-8.

The methods 400 and 500 are illustrated as process flow diagrams with flowchart illustrations. For purposes of simplicity, the methods 400 and 500 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 400 and 500. While not described in detail here, the multi-tiered reservoir(s) of the ophthalmic devices can include more than three tiers. The number of tiers can be one more than the number of sacrificial barriers positioned within the interior of each reservoir.

Referring now to FIG. 9, illustrated is a method 400 for controlling the release of two therapeutics from a two-tiered reservoir system of a smart ophthalmic device for treatment of a disease, disorder, or injury of the eye (e.g., glaucoma, dry eye, etc.). The smart ophthalmic device can include at least one two-tiered reservoir having an opening to an interior of the reservoir covered by a metal electrode and can be positioned on or in the eye of a subject. The multi-tiered reservoir can include a sacrificial barrier layer that can form two tiers within the interior of the reservoir, where one tier can hold a first therapeutic and the other tier can hold a second therapeutic. In one non-limiting example, the first and second therapeutics can be a drug and a second drug. At 402, the metal electrode covering the opening of the reservoir can be at least partially electrodissolved by the application of an electrical signal to the metal electrode. The electrical signal can be applied by a signal generator that can be controlled on demand and/or based on one or more sensed physiological parameters. At 404, a drug, or another type of therapeutic, can be released from the opening of the reservoir. The release of the drug can be by passive diffusion or can be actively facilitated, such as by iontophoresis. The released drug can move out of the reservoir and out of the ophthalmic device into an eye, or a target tissue of the eye.

At 406, the sacrificial barrier layer holding the second drug in the reservoir can be sacrificed. In one non-limiting example, the sacrifice can be water mediated where the sacrificial barrier layer is a water soluble material that facilitates the sacrifice when the tears from the eye enter the reservoir. The water soluble material can include, but is not limited to, a polymer and/or a salt. In another non-limiting example, the sacrifice can be electrically activated and/or mediated. The sacrificial barrier layer can be electrodissolvable, for example, the sacrificial barrier layer can include a metal configured for electrodissolution (such as a thin gold film), one or more electroresponsive polymers, and/or one or more electrothermal films. An electrical signal can be applied from the same signal generator used to electrodissolve the metal electrode covering the opening of the reservoir. The signal generator can be attached to a controller (e.g., computer, smartphone, a smart wearable accessory, etc.) that can communicate a start time and parameters of the electrical signal being applied to the electrical signal to electrodissolve the metal electrode and/or the sacrificial barrier layer. The sacrifice of the sacrificial barrier layer can be at a predetermined time after the electrodissolution of the metal electrode. At 408, the second drug can be released from the opening of the reservoir after the sacrifice of the sacrificial barrier layer. The second drug can be the same as the first drug or different. The released drug can move out of the reservoir and out of the ophthalmic device into an eye, or a target tissue of the eye. The release profile of the second drug can be based on a thickness (L) of the sacrificial barrier layer and/or timed by the controller in the case of electrically mediated sacrifice. The thicker the sacrificial barrier layer the longer sacrifice/dissolution takes.

Referring now to FIG. 10, is a method 500 for controlling the release of three therapeutics from a two-tiered reservoir system of a smart ophthalmic device for treatment of a disease, disorder, or injury of the eye (e.g., glaucoma, dry eye, etc.). The smart ophthalmic device can include at least one three-tiered reservoir having an opening to an interior of the reservoir covered by a metal electrode and can be positioned on or in the eye of a subject. The three-tiered reservoir can include two sacrificial barrier layers that can form three tiers within the interior of the reservoir, where one tier can hold a first therapeutic, a second tier can hold a second therapeutic, and a third tier can hold a third therapeutic with a sacrificial barrier layer between the first and second tiers and another sacrificial barrier layer between the second and third tiers. In one non-limiting example, each of the therapeutics can be a drug, which can be the same or different drugs, in any combination. At 502, the metal electrode covering the opening of the reservoir can be at least partially electrodissolved by the application of an electrical signal to the metal electrode. The electrical signal can be applied by a signal generator that can be controlled on demand and/or based on one or more sensed physiological parameters. At 504, a drug, or another type of therapeutic, can be released from the opening of the reservoir. The release of the drug can be by passive diffusion or can be actively facilitated, such as by iontophoresis. The released drug can move out of the reservoir and out of the ophthalmic device into an eye, or a target tissue of the eye. The first drug and the second drug may interact in a combinatorial manner after both have been released (before or after complete diffusion out of the ophthalmic device into/one the eye) to treat the disease, disorder, or injury of the eye.

At 506, the sacrificial barrier layer holding the second drug in the reservoir can be sacrificed. In one non-limiting example, the sacrifice can be water mediated where the sacrificial barrier layer is a water soluble material that facilitates the sacrifice when the tears from the eye enter the reservoir. The water soluble material can include, but is not limited to, a polymer and/or a salt. In another non-limiting example, the sacrifice can be electrically activated and/or mediated. The sacrificial barrier layer can be electrodissolvable, for example, the sacrificial barrier layer can include a metal configured for electrodissolution (such as a thin gold film), one or more electroresponsive polymers, and/or one or more electrothermal films. An electrical signal can be applied from the same signal generator used to electrodissolve the metal electrode covering the opening of the reservoir. The signal generator can be attached to a controller (e.g., computer, smartphone, a smart wearable accessory, etc.) that can communicate a start time and parameters of the electrical signal being applied to the electrical signal to electrodissolve the metal electrode and/or the sacrificial barrier layer. The sacrifice of the sacrificial barrier layer can be at a predetermined time after the electrodissolution of the metal electrode. At 508, the second drug can be released from the opening of the reservoir after the sacrifice of the sacrificial barrier layer. The second drug can be the same as the first drug or different. The released second drug can move out of the reservoir and out of the ophthalmic device into an eye, or a target tissue of the eye. The release profile of the second drug can be based on a thickness (L) of the sacrificial barrier layer and/or timed by the controller in the case of electrically mediated sacrifice. The thicker the sacrificial barrier layer the longer sacrifice/dissolution takes.

At 510, the second sacrificial barrier layer holding the third drug in the reservoir can be sacrificed. The sacrifice of the second sacrificial barrier can be water mediated and/or electrically mediated based on the composition of the second sacrificial barrier. The sacrifice of the second sacrificial barrier can be the same as or different than the first sacrificial barrier. The sacrifice of the second sacrificial barrier layer can be at a predetermined time after the sacrifice of the first sacrificial barrier layer. At 512, the third drug can be released from the opening of the reservoir after the sacrifice of the second sacrificial barrier layer. The third drug can be the same as the first drug and/or the second drug or different than both the first and second drugs. The released third drug can move out of the reservoir and out of the ophthalmic device into an eye, or a target tissue of the eye. The release profile of the third drug can be based on a thickness (L) of the second sacrificial barrier layer and/or timed by the controller in the case of electrically mediated sacrifice. The thicker the second sacrificial barrier layer the longer sacrifice/dissolution takes. At least two of the first drug, the second drug, and the third drug may interact in a combinatorial manner after both have been released (before or after complete diffusion out of the ophthalmic device into/one the eye) to treat the disease, disorder, or injury of the eye.

From the above description, those skilled in the art will perceive improvements, changes, and modifications. Such improvements, changes and modifications are within the skill of one in the art and are intended to be covered by the appended claims.

The following is claimed:

1. An ophthalmic device comprising:
a reservoir having an interior, wherein the interior comprises a first drug, a sacrificial barrier layer, and a second drug; and
a metal electrode configured to cover an opening of the reservoir and to receive an electrical signal that electrodissolves the metal electrode,
wherein the first drug is released upon electrodissolution of the metal electrode and the second drug is released upon sacrifice of the sacrificial barrier layer.

2. The ophthalmic device of claim 1, wherein the sacrificial barrier layer is sacrificed a predetermined time after the electrodissolution of the metal electrode.

3. The ophthalmic device of claim 1, wherein the sacrificial barrier layer is a water soluble material to facilitate the sacrifice.

4. The ophthalmic device of claim 3, wherein the water soluble material comprises a polymer and/or a salt.

5. The ophthalmic device of claim 1, wherein the sacrificial barrier layer is configured to be electronically activated to facilitate the sacrifice.

6. The ophthalmic device of claim 5, wherein the sacrificial barrier layer comprises a metal configured for electrodissolution.

7. The ophthalmic device of claim 5, wherein the sacrificial barrier layer comprises one or more electroresponsive polymers and/or one or more electrothermal films.

8. The ophthalmic device of claim 1, further comprising a body comprising a hydrogel-based material configured to encapsulate the reservoir and the metal electrode.

9. The ophthalmic device of claim 1, wherein the interior further comprises a second sacrificial barrier layer and a third drug,
wherein the third drug is released upon sacrifice of the second sacrificial barrier layer.

10. The ophthalmic device of claim 9, wherein the second sacrificial barrier layer is sacrificed at a second predetermined time after the sacrificial barrier layer is sacrificed.

11. The ophthalmic device of claim 10, wherein the predetermined time is based on a thickness of the second sacrificial barrier layer.

12. The ophthalmic device of claim 1, wherein a release profile of the second drug is based on a thickness of the sacrificial barrier layer.

13. A method comprising:
electrodissolving a metal electrode covering an opening of a reservoir of an ophthalmic device;
releasing a drug from the opening of the reservoir;
sacrificing a sacrificial barrier layer holding a second drug in the reservoir; and
releasing the second drug from the opening of the reservoir.

14. The method of claim 13, wherein a release profile of the second drug is based on a thickness of the sacrificial barrier layer.

15. The method of claim 13, wherein the sacrificial barrier layer is sacrificed a predetermined time after the electrodissolution of the metal electrode.

16. The method of claim 13, wherein the sacrificial barrier layer is a water soluble material to facilitate the sacrifice.

17. The method of claim 16, wherein the water soluble material comprises a polymer and/or a salt.

18. The method of claim 13, wherein the sacrificial barrier layer is configured to be electronically activated to facilitate the sacrifice.

19. The method of claim 18, wherein the sacrificial barrier layer comprises a metal configured for electrodissolution.

20. The method of claim 18, wherein the sacrificial layer comprises one or more electroresponsive polymers and/or one or more electrothermal films.
